(19) United States
(12) Patent Application Publication
(10) Pub. No.: US 2018/0070901 A1
(43) Pub. Date: Mar. 15, 2018

(54) Radiography Appliance Comprising a Movable Arm

(71) Applicant: ECOLE POLYTECHNIQUE FEDERALE DE LAUSANNE (EPFL), Lausanne (CH)

(72) Inventors: Thomas Gabella, Lausanne (CH); Carlo Mentano, Yverdon-les-Bains (CH)

(21) Appl. No.: 15/554,735

(22) PCT Filed: Mar. 7, 2016
(86) PCT No.: PCT/IB2016/051271

§ 371 Date: Aug. 31, 2017

(30) Foreign Application Priority Data
Mar. 6, 2015 (IB) PCT/IB2015/051659

(51) Int. Cl. A61B 6/00

(52) U.S. Cl. CPC A61B 6/4452; A61B 6/102; A61B 6/4435

(57) ABSTRACT

The invention relates to a radiography appliance comprising a column and a movable arm mounted such that it slides along the column and rotates about an axis oriented perpendicularly to the main direction of the column, one end of the arm comprising a detector and the other end a radiation source. The appliance is characterised in that it comprises means for locking the arm against rotation and/or against translation.

This application claims the benefit of international application No. PCT/IB2015/051659 filed on Mar. 6, 2015, the entire content of which is incorporated by reference into this application.

### FIELD OF THE INVENTION

The invention relates to radiography appliances, particularly X-ray radiography appliances.

It relates more specifically to radiography appliances in which the radiation source and the detector are positioned at the ends of a mobile arm.

### PRIOR ART

In most X-ray radiography appliances a mobile arm is mounted with the ability to slide along a vertical column the base of which is fixed to the floor. The mobile arm may also rotate about an axis oriented perpendicular to the main direction of the column.

The combination of the translational and rotational movements of the arm may cause the latter to impact with the floor, something which is not desirable, particularly because of the sensitivity of the source and of the detector which are located at the ends of the arm.

Aside from the impacts caused by the aforementioned combined movements, the relatively high weight of the arm requires a relatively large amount of effort on the part of the user when the latter needs to move it. The high inertia generated by its movement also increases the risk of not being able to halt the movement of the arm soon enough, particularly soon enough to avoid impact with the ground.

An impact may also occur when the source is being moved along the arm. Such a movement alters the distribution of total weight along the arm, and this may result in accidental rotation of the arm and therefore in one of its ends impacting with the floor.

There is therefore a need to avoid the abovementioned impacts.

### SUMMARY OF THE INVENTION

The present invention notably allows the aforementioned problems to be remedied.

The present invention makes it possible to remedy the problems mentioned in the previous section and relates to a radiography appliance according to claim 1.

Other advantageous features of the invention are indicated in the dependent claims.

In general, it relates to a medical imaging appliance, more specifically a radiography appliance as defined in the claims, which comprises a support column and an arm capable of moving and articulated to the column or mounted with the ability to slide along the column. The arm includes a first support device able to bear a radiation source and a second support device able to bear a radiography detector, the first or the second device being capable of moving along the arm. The appliance further comprises anti-rotation means configured to limit or prevent rotation of the arm with respect to the column and/or braking means configured to slow the movement of the arm along the column so as to avoid a collision of the arm with the floor.

The appliance according to the invention makes it possible to limit the overall movement of the arm in a very simple way, with no external system placed on the floor or the ceiling and without excessively limiting the movement.

In addition, the existing systems are complex and usually constructed in such a way that the source and the detector move simultaneously or that a counterweight system is activated so that the moment generated by the eccentric weight of the system remains small and rotation of the arm toward the ground is avoided.

The system proposed by the present invention makes it possible, mechanically and reliably, to ensure that the source can be moved away from the detector only when the arm is in a horizontal position rotation is mechanically blocked by impediment during the distancing movement of the source.

A complementary system based, for example, on the use of a roller and of a guide makes it possible to ensure a play-free and smooth transition between the authorized rotation and extension movement of the arm. The invention will be better understood hereinafter by means of examples. The invention is not limited to these examples.

### BRIEF DESCRIPTION OF THE FIGURES

Further features and advantages of the present invention will become more clearly apparent from reading the detailed description which stems from one embodiment of the invention, which embodiment is given by way of entirely nonlimiting example and illustrated by the attached drawings, in which:

FIG. 1 illustrates a radiography appliance according to the invention, comprising means for avoiding impacts between the system and the ground;

FIG. 2 depicts a radiography appliance according to the invention illustrating the positions occupied by the arm during a tilting movement which are allowed by the blocking means present;

FIG. 3a illustrates a first situation in which the arm is as close as possible to the ground and in which its rotation is prevented by the blocking means so that the arm will not collide with the ground;

FIG. 3b illustrates a second situation in which the arm is as far away from the ground as possible and in which its rotation is possible toward the illustrated position is possible;

FIG. 3c illustrates a second situation in which the arm is in an intermediate position with respect to the ground and in which its rotation is prevented in part by the blocking means so that the arm will not collide with the ground;

FIG. 3d is a view in section on A-A of FIG. 3a of the appliance illustrated in FIG. 3a;

FIG. 4 illustrates a one-way braking mechanism of the present invention, configured to brake/block the movement of the arm along the column;

FIG. 5 illustrates a two-way braking system of the present invention configured to brake/block the movement of the arm along the column;

FIG. 6 depicts a view in section of the braking system of FIG. 5;

FIG. 7 illustrates another aspect of the present invention and a radiography appliance according to the invention, particularly the distancing movement of a source away from a detector is illustrated;

FIG. 8 depicts a view in section of an arm of the imaging appliance according to the invention and a system for blocking the distancing movement of the source and the rotation of the arm in a situation in which the arm is horizontal and in which the distancing movement of the source is permitted;

FIG. 9 illustrates a view in section of an arm of the imaging appliance according to the invention and the system for blocking the distancing movement of the source and the rotation of the arm in a situation in which the arm is not horizontal and in which the distancing movement of the source is not permitted;

FIG. 10 is another view in section of the arm of an imaging appliance according to the invention and the system for blocking the distancing movement of the source and the rotation of the arm in a situation in which the arm is not horizontal and in which the distancing movement of the source is not permitted;

FIG. 11 depicts a view in section of the arm of an imaging appliance according to the invention and the system for blocking the distancing movement of the source and the rotation of the arm in a situation in which the arm is horizontal and in which the distancing movement of the source is permitted;

FIG. 12 depicts a view in section of the arm of an imaging appliance according to the invention in a situation identical to the one illustrated in FIG. 11, but with also the blocking of the rotation of the arm;

FIG. 13 illustrates a view in section of the arm of an imaging appliance according to the invention in a horizontal position in which the source has moved in order to distance it from the detector (movement along the straight line in the figure) and in which the rotation is blocked by the blocking system; and FIG. 14 is a face-on view of the arm shown in FIG. 13.

### DETAILED DESCRIPTION OF THE INVENTION

Mechanism controlling the combined rotational and translational movements of the arm

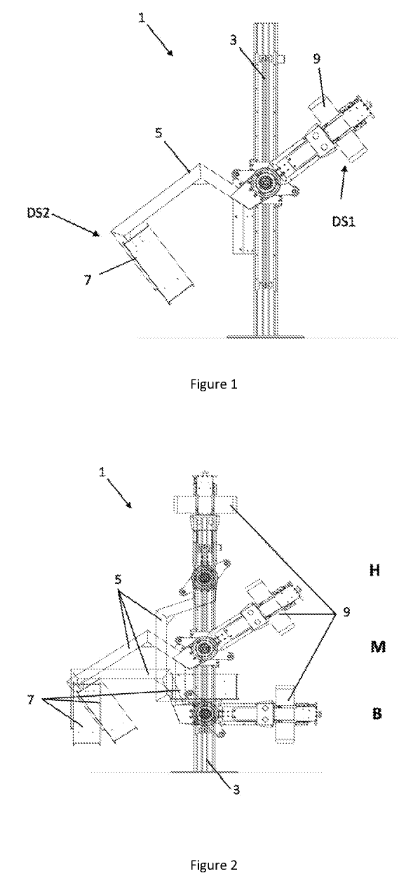

FIG. 1 illustrates the radiography appliance (or a medical imaging appliance) 1 according to the invention. The appliance 1 comprises a support column 3, an arm 5 mounted with the ability to move and articulated on the column 3. The arm 5 being able to slide along the column 3 and to rotate about an axis oriented perpendicular to the direction of the column 3.

The arm 5 further comprises a first support device DS1 able to bear a radiation source 7 on a first side of the arm and a second support device DS2 able to bear a detector 9 on a second side of the arm. The first or the second support device are able to move and can move along the arm 5.

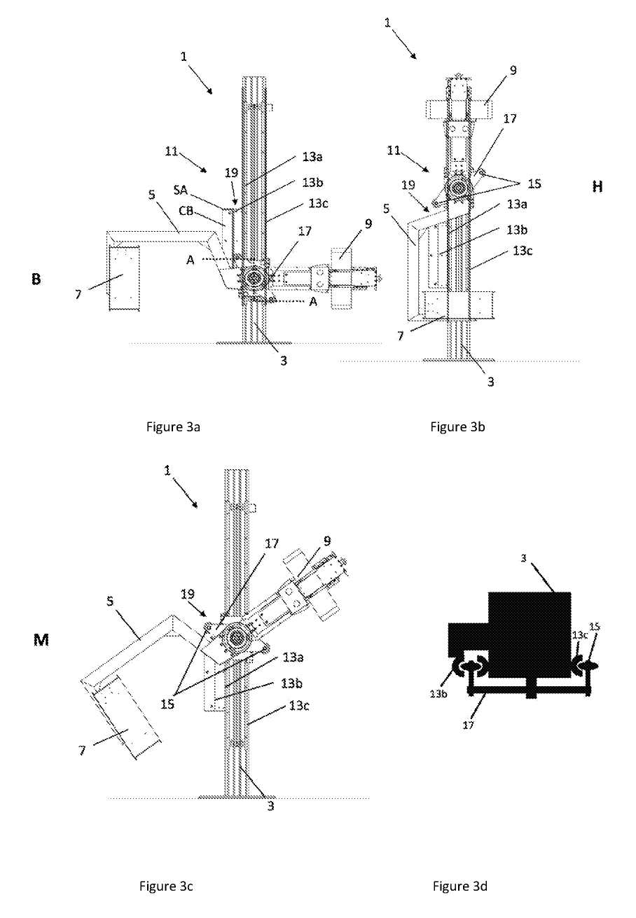

The radiography system 1 additionally comprises anti-rotation means 11 (see FIG. 3) configured to limit or prevent the rotation of the arm 5 with respect to the support column 3 so as to avoid collision between the arm and the ground and therefore avoid damage to the radiography appliance 1.

The anti-rotation means 11 are configured to differentially limit the rotation of the arm 5 according to the position thereof along the column 3.

FIG. 2 illustrates various positions of the arm and the various possible rotations of the arm 5 according to the position along the support column 3. When the arm is in a first lower position B, namely as close as possible to the base of the column 3 and therefore to the ground, the anti-rotation means 11 are configured to limit the rotation of the arm 5 with respect to the column 3, the arm being kept in a position substantially perpendicular to the column 3 and its authorized angle of rotation being very small, or even zero.

When the arm is in an upper position H, as close as possible to the top of the support column 3 and therefore as far away from the ground as possible, the anti-rotation means 11 limit the rotation of the arm 5 little if at all, this arm being able to rotate as far as a position substantially parallel to the column 3.

When the arm is in an intermediate position (M), which means to say a position between the lower position B and the upper position H, the anti-rotation means 11 are configured to limit the rotation of the arm 5 with respect to the column 3 so that the arm 5 does not come into contact with the ground when it rotates. Complete rotation of the arm 5 into a position substantially parallel to the column 3 is prevented by the anti-rotation means 11 although its authorized angle of rotation remains greater than in the lower position B but smaller than in the upper position H.

FIGS. 3a, 3b and 3c illustrate in greater detail the anti-rotation mechanism or means 11 of the first alternative form of the radiography appliance 1 according to the invention and the lower B, intermediate M and upper H positions.

As illustrated by FIG. 3a, the anti-rotation means 11 include a system made up of a first guide (or guide device) 13a, a second guide (or guide device) 13b, and a third guide (or guide device) 13c which are essentially arranged in a direction substantially parallel to the main direction of the column (substantially vertical with respect to the floor), the first guide 13a and the third guide 13c extending further than the second guide 13b over an upper part of the column and delimiting an angle of rotation of the arm in its upper position.

The first guide 13a and the third guide 13c are connected or fixed to the column 3. The second guide 13b is connected or fixed to the column 3 and kept a distance away from the first guide 13a by a column 3 extension body CB.

The anti-rotation means 11 (and the system) also comprise two rollers 15, the two rollers 15 being able to move along the guides 13a, 13b, 13c and the roller on the same side as the guide 13b can move between the guide 13a and 13b.

The guide 13a, 13b, 13c is, for example, a U-shaped channel (see FIG. 3d) able to receive the roller 15.

The rollers 15 are mounted at the ends of an element or member 17 fixed to the arm at the level of its axis of rotation on the column 3. The element or member 17 extends substantially in the continuation of the column so that the column lies between the two rollers 15.

The guides define the limits imposed on the movement of the rollers and therefore of the arm 5, the movement of the arm 5 is limited by contact between the rollers 15 and the guides 13a, 13b and 13c.

The limits on the movement of the arm 5 are also imposed in part by the column itself. Furthermore, a lower limit stop (not illustrated) blocks the movement of the arm at a minimum height.

The first guide 13a and the second guide 13b form a guide element 19 extending over a limited part of the column and allowing the movement of the arm to be guided between a lower position B (FIG. 3a) in which the rotation of the arm with respect to the column is limited and the arm is kept in a position substantially perpendicular to the column and an upper position H (FIG. 3b) in which the rotation of the arm into a position substantially parallel to the column is possible.

One element of the member 17, for example the roller 15, is received by the guide element 19 and guided thereby to guide the movement of the arm 5 between the lower position B and the upper position H.

The guide element 19 includes the first guide 13a extending along the column 3 over a first distance and the second guide 13b extending along the column over a second distance shorter than the first distance. Rotation of the arm is limited by contact between the roller 15 and the first guide 13a and the roller 15 and the second guide 13b.

The guide element 19 may further include the aforementioned lower limit stop. The limit stop may, for example, be situated at the lower end of the guide element 19 (not illustrated).

In FIG. 3a may be seen the guide 13b forming the guide element 19 including an angular section SA making it possible to increase the rotation of the arm. For example, the guide 13b is in the shape of an inverted L. The angle of the L corresponds to the upper limit of a straight-line segment or of the guide element 19 the other end of which is, for example, the aforementioned lower limit stop (not illustrated). When it lies in this segment, the arm remains in a substantially horizontal position; it cannot be pivoted (FIG. 3a) or can be pivoted only a little. When it emerges from this segment and the roller 15 is resting on the short branch of the L, the arm can rotate counterclockwise. However, this rotation is limited by the short branch of the L of the guide 13b which acts as a limit stop (FIG. 3c). Finally, above and beyond a certain arm height (see FIG. 3b), when the roller no longer comes into contact with the short branch of the L, the angle of rotation of the arm can be maximized. In this configuration, the arm can be oriented parallel to the column.

The control mechanism or the anti-rotation system illustrated in FIGS. 3a, 3b and 3c therefore makes it possible to avoid contact between the arm and the ground.

The anti-rotation system illustrated in FIGS. 3a to 3c comprises the first guide (or guide device) 13a, the second guide (or guide device) 13b, and the third guide (or guide device) 13c. However, the anti-rotation system can include only the first guide (or guide device) 13a and the second guide (or guide device) 13b. The anti-rotation system can include only the guide element 19 and furthermore there is no need for the first guide 13a to extend fully along the column 3. The first guide 13a may stop at the same height as the second guide. The anti-rotation means (or system) 11 are thus configured to limit or prevent the rotation of the arm 5 over the limited part of the column 3 comprising such a the guide element 19.

Braking and blocking the arm in terms of translational movement

The arm 5 (FIG. 1), which may weight around a hundred kg or more, needs to be able to be moved vertically without effort and stopped stably with a minimum of control effort. The system also needs to be compact.

The solution proposed by the present invention is preferably based on use of two one-way self-blocking systems placed in opposition and preloaded.

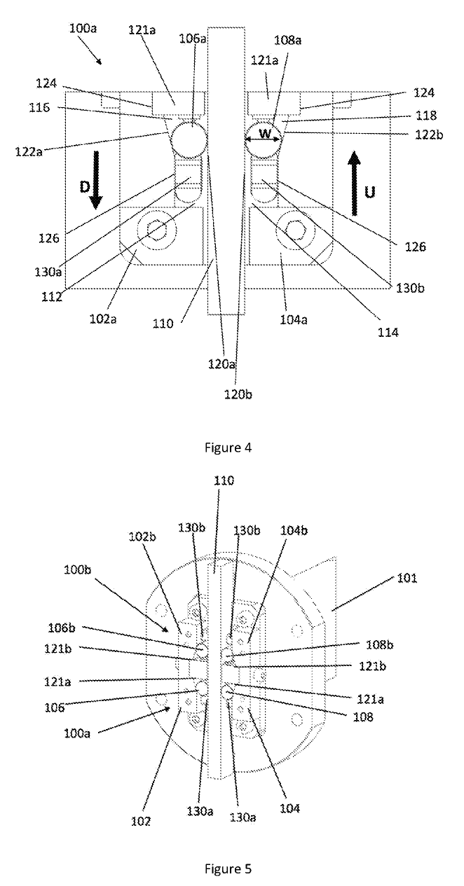

According to another aspect of the present invention, the radiography appliance comprises braking means (or a braking mechanism) comprising the one-way system 100a (FIGS. 4 and 5).

The one-way system 100a is, for example, mounted on a connecting arm (or fixed pivot) 101 of the column (FIG. 5) that connects the column 3 of the radiography appliance to the arm 5 of the radiography appliance.

The one-way system 100a includes a first block 102a, a second block 104a and a first friction body 106a and a second friction body 108a (for example a roller of cylindrical shape).

The first 102a and the second 104a blocks are situated on each side of the rail 110 of the column 3 of the radiography appliance, each block delimiting a braking passage or channel 112, 114 and a preloading passage or channel 116, 118.

A wall 120a, 120b of the rail 110 and a wall 122a, 122b of the block delimit the braking passage 112, 114 and the preloading passage 116, 118. The wall 122a, 122b of the block comprises an upper part 124 and a lower part 126, the upper part having a steeper slope than the lower part. The wall of the rail and the upper part 124 of the wall 120a, 120b delimit the preloading passage 116, 118. The wall of the rail and the lower part 126 of the wall 120a, 120b delimit the braking passage 112, 114.

The first friction body 106a is situated in the first preloading passage 116 and the second friction body 108a is situated in the second preloading passage 118.

The system 100a comprises a preloading device or system 121a (for example involving a spring) applying a preloading force (in the direction D) to each friction body 106a, 108a in order to push each friction body toward the outside of the preloading passage 116, 118 and toward the braking passage 112, 114.

The preloading device 121a ensures self-blocking braking.

The preloading device 121a applies a force to the friction body 106a, 108a making it possible to ensure that the system 100a is free of play.

The system 100a comprises means of applying a force which are configured to apply a force in an opposite direction (in the direction U) to the direction of the preloading force on the friction bodies 106a, 108a, when the arm is moved along the rail 110 in the direction D down the rail 110 toward the ground.

In the example illustrated in FIG. 4, (a) the contact between the wall 122a, 122b of the preloading passage and the friction body and/or (b) the contact between the surface 120a, 120b of the rail and the friction body form these means of applying a force, applying a force in a direction the opposite of the direction of the preloading force or in the same direction as the preloading force.

This force is greater than the preloading force so as to allow the arm to move in a single direction, for example the downward direction in FIG. 4.

The means of applying a force are configured to apply a force in the same direction of the preloading force (in the direction D) to the friction bodies when the arm is moved along the rail 110 in a direction U up the rail 110.

The braking passage is smaller than the preloading passage. Each friction body and each passage has a width in a direction substantially perpendicular to the rail 110 and substantially parallel to the arm.

Each friction body has a width W substantially equal to or greater than a width of the braking passage 112, 114. Each friction body has a width W smaller than a width of the preloading passage 116, 118.

The preloading device 121a may apply a force to each friction body so as to keep the arm blocked on the rail.

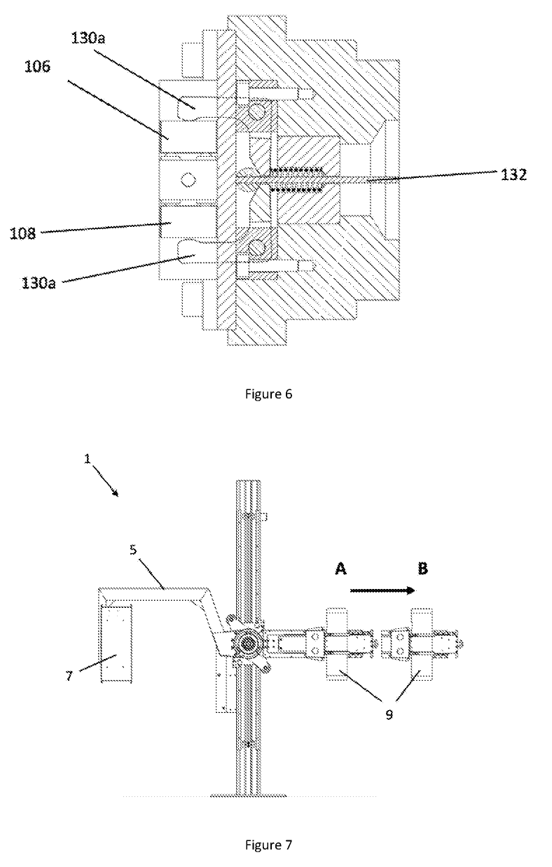

The system 100a may comprise a pair of unblocking levers 130a, 130b (FIGS. 5 and 6) collaborating with each friction body 106a, 108a so as to move each friction body toward the inside of the preloading passage and out of the braking passage.

The system 100a further comprises a control mechanism 132 connected to the pair of levers 130 in order to actuate these (FIG. 6). FIG. 6 illustrates a system for unblocking the rollers by levers actuated by a cable controlling the unblocking.

Each one-way system (FIG. 4) comprises, for example, a pair of cylindrical rollers sandwiched between a V-shaped caliper and a rail. The angle of the pressing V is dimensioned so that the rail/cylinders system is self-blocking when the caliper is loaded in the direction away from the point of the V. In the one-way system of FIG. 4, the caliper can move in the direction D and is prevented from moving in the direction U.

A rollers pre-loading system actuated by springs makes it possible to ensure that the system is free of play.

According to another alternative form of the present invention, illustrated in FIG. 5, the braking means comprise a second one-way system 100b situated above the first one-way system 100a so as to allow two-way braking.

The first 100a and the second 100b one-way systems are arranged symmetrically on each side of the rail 110 (see FIG. 5).

The first 100a and the second 100b one-way systems are placed in opposition allowing the movement of the arm along the column to be slowed and/or blocked.

The second one-way system 100b is identical to the first one-way system 100b. The second one-way system 100b includes a first block 102b, a second block 104b and a first friction body 106b and a second friction body 108b (FIG. 5).

The first 102b and the second 104b blocks are situated one on each side of the rail 110 of the column of the radiography appliance, each block delimiting a braking passage or channel and a preloading passage or channel.

The second system 100b includes a second preloading device 121b applying a preloading force (in the direction U) to each friction body so as to push each friction body toward the outside of the preloading passage and toward the braking passage.

The second preloading device 121b applies a preloading force in a direction U the opposite of the direction D of the force applied by the first preloading device 121a (FIG. 5).

Each preloading device 121a, 121b may apply a force to each friction body so as to keep the arm blocked on the rail 110.

A pair of levers 130b collaborating with each friction body 106b, 108b of the second system 100b so as to move each friction body toward the inside of the preloading passage and out of the braking passage thus releasing the arm.

The control mechanism 132 is also connected to the pair of levers 130 in order to actuate same.

The second system 100b further comprises means of applying a force which are configured to apply a force in an opposite direction (in the direction D) to the direction of the preloading force to the friction bodies when the arm is moved along the rail in the direction U.

The means of applying a force are also configured to apply a force in the same direction as the preloading force (in the direction U) to the friction bodies when the arm is moved along the rail in the direction D.

The two-way blocking of the complete brake is provided by the coupling of two one-way systems placed in opposition and kept blocked by the common spring-loaded preloading system as shown in FIG. 5.

The unblocking of the brake is actuated by the pair of levers, which are, for example, activated by a cable as shown in FIG. 6. A pull on the cable causes the levers to pivot and push the rollers back toward one another, thus eliminating the blocking.

System excluding simultaneous rotation and extension movement of the arm

As illustrated in FIG. 7, the radiography system or appliance 1 of the present invention is designed so that the ray source 9 can be distanced from the detector 7 when the arm 5 is in a horizontal position between a position A and a position B (see FIG. 7).

According to yet another aspect of the present invention, the system proposed by the present invention makes it possible mechanically and reliably to ensure that
the source can be distanced from the detector only when the arm is in a horizontal position
rotation is mechanically blocked by impediment during the distancing movement of the source.

A complementary system based, for example, on the use of a roller and of a guide makes it possible to ensure a play-free and smooth transition between the authorized rotational and extensional movement of the arm.

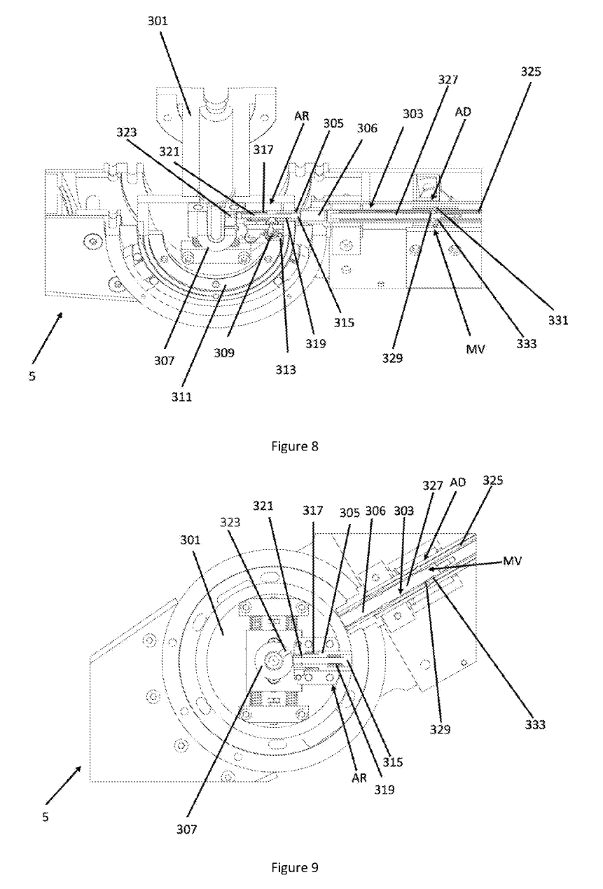

The appliance 1 is similar to the appliance of FIG. 1 and includes a column 3 comprising a connecting arm or a fixed pivot 301 (see FIG. 8) mounted on the column 3. This connecting arm or fixed pivot is also visible in FIG. 5.

The arm 5 of the appliance is connected to the column 3 (not illustrated) by the fixed pivot 301. The arm 5 of the appliance is mounted with the ability to rotate on the pivot 301 about an axis substantially perpendicular to the column 3.

FIG. 8 depicts the main elements of a mechanism or system included by the radiography appliance 1 and this mechanism or system being configured to block the movement of the first support device DS1 or of the second support device DS2 of the arm 5 depending on the inclination of the arm 5 and to block the rotation of the arm 5 when the first or second support device is in motion.

The mechanism comprises an anti-rotation device AR to prevent the rotation of the arm 5 with respect to the column 3. It further comprises an anti-displacement device AD to prevent the first DS1 or second DS2 support device from moving along the arm 5.

The anti-rotation device AR is included in the arm 5 and the column 3. The anti-displacement device AD is incorporated into the arm 5.

The arm 5 further comprises an actuator 303 configured to activate the anti-rotation device AR and to deactivate the anti-displacement device AD so as to allow the first DS1 or second DS2 support device to move along the arm 5 without rotation of the latter.

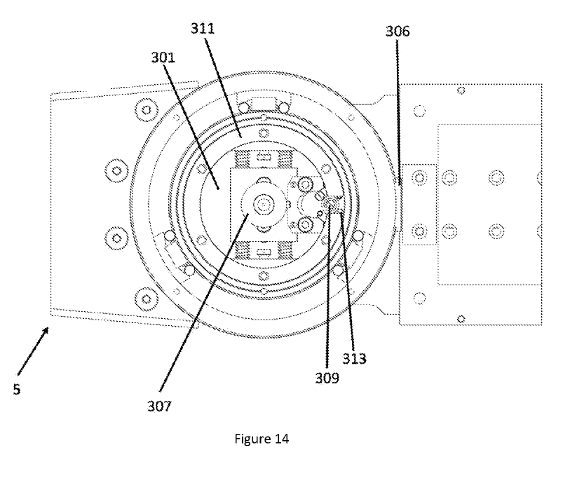

The anti-rotation device AR comprises a blocking member 305 (or indexing finger) mounted with the ability to move or slide between a first rotation blocking position illustrated in FIG. 7, in which at least part of the blocking member 305 is received by the arm 5 (see FIG. 13) and at least part of the blocking member 305 is simultaneously received by part of the column (for example the fixed pivot 301) and a second rotation non-blocking position in which the blocking member 305 is housed entirely in part of the column (for example in the fixed pivot 301 as illustrated in FIG. 13).

The arm 5 comprises a guide body 306 (or indexing socket) mounted in the arm 5 at the gap with the column (or fixed pivot 301). The guide body 306 is able to receive the blocking member 305 and the actuator 303 when the arm 5 is in a position substantially perpendicular to the column 3. The guide body 306 comprises a cylindrical internal cavity with an internal diameter capable of receiving the blocking member 305 and/or the actuator 303.

When the blocking member 305 is in the first unblocking position, the arm 5 is kept in a position substantially perpendicular to the column 3.

The blocking member 305 is constantly pushed outward (to the right in FIG. 8) by a return spring 317. When it is in the position shown in FIG. 8 (in an advanced position in the arm 5) the blocking member 305 is the component that allows the arm 5 and the pivot 301 or the column to be secured to one another in terms of rotation.

The sliding movement of the blocking member 305 in the column 3 and in the arm 5, more specifically in the guide body 306, is limited to the left in FIG. 8 by an indexing spindle 307 and to the right by a retaining element 309, for example a retaining roller.

The retaining element 309 is mechanically connected to the blocking element 305, for example by means of a pin and allows the blocking member 305 to be kept in the second non-blocking position as the arm 5 rotates.

The retaining element 309 presses against a retaining cam 311 capable of collaborating with the retaining element 309 in order to guide same.

The retaining cam 311 is circular in shape corresponding to the shape of the fixed pivot 301. The cam 311 is placed inside this pivot while at the same time being secured to the arm 5. As a result, when the arm 5 is rotating, the retaining cam 311 follows the same rotational movement. The retaining cam 311 is in contact with the retaining element 309 (the roller 309).

The retaining cam 311 includes an opening 313 to guide the retaining element 309 and, therefore, the blocking member 305, toward the first position of blocking the rotation of the arm 5 as illustrated in FIG. 8. The opening 313 corresponds to the situation in which the arm is horizontal or in a direction substantially perpendicular to the column 3.

The blocking member 305 further comprises a blocking element 315, for example an indexing spindle. The blocking element 315 is mounted with the ability to move in the blocking member 305 between a first position of blocking the rotation, in which position at least part of the blocking element 315 is held in the blocking member 305 and at least part of the blocking element 315 is simultaneously received by the arm 5 as illustrated in FIG. 8, and a second non-blocking position in which the blocking element 315 is housed entirely in the blocking member 305 as illustrated in FIG. 9.

The blocking element 315 is constantly pushed outward (to the right in FIG. 8) by a spring 319. Its longitudinal movement is limited outward (to the right in FIG. 8) by a circlip 321 which prevents the blocking element 315 from coming out of the blocking member 305, and toward the inside (to the left in FIG. 8) by the indexing spindle 307. The pin 315 can therefore be pushed inward (to the left in the figure) only when the pin 315 is facing the hole made horizontally in the indexing spindle 307.

The pin 315 can therefore be pushed inward (to the left in FIG. 8) only when the pin 315 is facing a hole 323 made horizontally in the indexing spindle 307, the hole having a diameter that allows the pin 315 to slide partly into this hole.

The blocking element 315 faces the hole 323 in the indexing spindle when the arm 5 is in a position or in a direction substantially perpendicular to the column 3.

The actuator 303 is mounted with the ability to move in the arm 5 to act on the anti-rotation device AR so as to activate and deactivate same. More specifically it allows the blocking member 305 to move between its blocking position and its non-blocking position.

The actuator 303 includes a control tube 325 mounted with the ability to move in the arm and a control rod 327 mounted with the ability to move in the control tube 325 and therefore in the arm 5 allowing the blocking element 315 to be moved between its blocking position and its non-blocking position.

The actuator 303 is further configured to deactivate the anti-rotation device AR and activate the anti-displacement device AD so as to allow the arm 5 to rotate without movement the support device supporting, for example, the source 9.

The anti-displacement device AD comprises the control tube 325 which is secured to the support device bearing the radiation source 9. The anti-displacement device AD further comprises the control rod 327 mounted with the ability to move in the control tube 325.

The control rod and the control tube may be moved radially along the arm. The control tube moves together with the support device or carriage holding the source 9. When the control tube moves to the right in the figures, the source is distanced from the radiation detector positioned on the other wing of the arm 5.

The anti-displacement device AD also comprises locking means MV configured to prevent the translational movement of the control tube 325 and therefore the translational movement of the ray source 9 attached securely to the control tube.

The locking means MV comprise at least one locking element 329. The locking element 329 for example comprises a plurality of (for example two) balls.

The locking element 329 is able to move on the outside of the control tube 325 and of the anti-displacement device AD so as to secure the control tube 325 and the anti-displacement device to the arm 5.

The arm further comprises a recess 331 able to receive the locking element 329 (for example the balls 329) or any other suitable locking element. The recess 331 is, for example, a toroidal notch or groove present in the arm 5.

The recess 331 is able to receive the locking element 329, for example the balls 329.

The movement of the locking element 329 in the recess 331 through the movement of the control rod 327 in the control tube 325 allows the control tube 325 to be dissociated from the arm 5. As a result, the support device bearing the source 9 is released and can move along the arm 5 and the source 9 can be moved translationally.

The control rod 327 comprises a declination or cam profile 333 on its surface. This profile collaborates with the locking element 329 to move the latter, for example out of the control tube 325 so as to lock the movement of the control tube and therefore of the source 9, or into the control tube 325 so as to unlock the movement of the control tube and therefore of the source 9.

For preference, the cam profile 333 is machined on a surface of the control rod 327 to release the locking element 329 when the rod is moved toward the center of the arm 5 so as to dissociate the control tube from the arm 5 and allow the support device bearing the source 9 (or the detector 7) to be unblocked.

It is also conceivable for a system as described hereinabove to be applied to the second support device, so as to lock/unlock the movement of the detector 7.

The control rod 327 can slide slightly in the control tube 325. The limit on the relative movement between the rod and the control tube is fixed, for example, by the “quick-coupling” balls 329 which press against the cam profile machined in the rod.

Movement of the control tube 325 allows the anti-displacement device AD to be activated and deactivated.

The actuator 303 is also configured to deactivate the anti-rotation device AR and to activate the anti-displacement device AD so as to allow the arm to rotate without movement of the first or second support device along the arm.

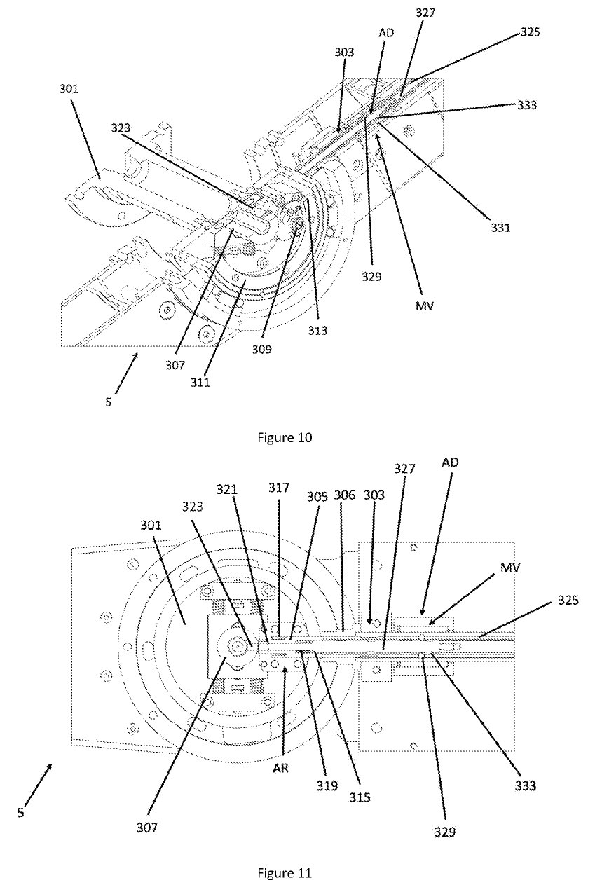

The “rotation possible” and “distancing movement of the source impossible” position is illustrated in FIGS. 9 and 10 and is characterized in that the arm 5 is not in a horizontal position or is not substantially perpendicular to the column. The indexing finger or blocking member 305 is blocked inward by the indexing spindle 307 and outward is retained by the retaining roller or the retaining element 309 which presses against the retaining cam 311. Without the action of the retaining roller, the indexing finger would have the possibility of exiting its housing in an outward direction.

In this position, the control rod 327 cannot be actuated because it presses against the fixed pivot 301 (or against the column). Because the control rod 327 cannot be moved further inward, the control tube 325 cannot be manipulated. In effect, the control rod 327 and the control tube 325 are secured to the arm 5 by means of the “quick coupling” balls 329 which are captive or housed in the recess, preferably a toroidal notch made in the arm 5. The system works like a hydraulic quick coupling.

The control tube 325 and therefore the carriage or support device bearing the source 9 cannot be set in motion if the control rod 327 is not pushed toward the center of the pivot in order to disengage the balls 329 by virtue of the cam profile 333 machined on the control rod 327 allowing the balls 329 to be disengaged from the toroidal notch 331. Extension movement of the arm is thus blocked.

FIGS. 11, 12, 13 and 14 illustrate the “rotation impossible” and “distancing movement of the source possible” position. In this situation, the system is in a horizontal position in which the arm is substantially perpendicular to the column. FIGS. 11, 12, 13 and 14 illustrate actuation of the blocking member (indexing finger) 305 and the unblocking and movement of the assembly made up of the control tube 325/carriage of the source 9.

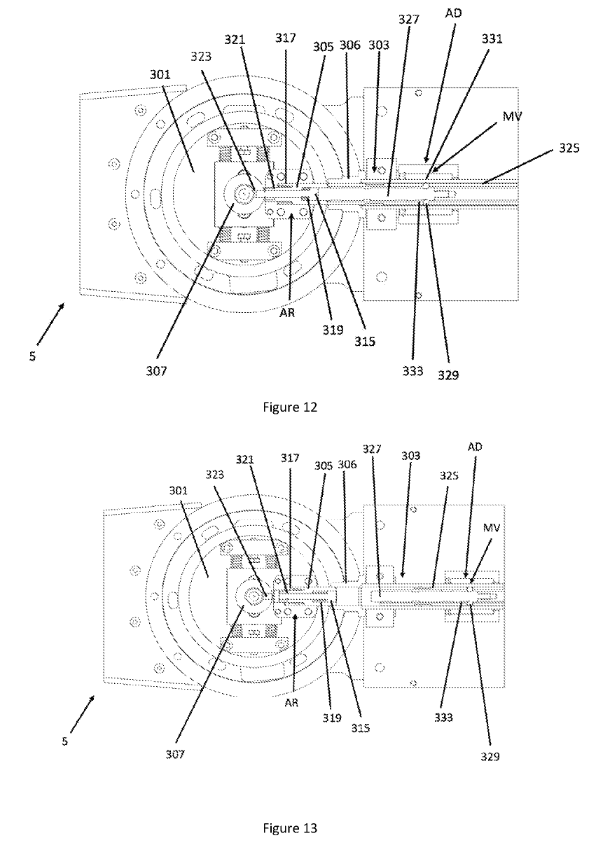

As FIG. 11 shows, when the system is in a horizontal position, the blocking element (the indexing pin) 315 lies facing the hole 323 placed in the indexing spindle 307. In this way, the control rod 327 can be pushed further in by pushing, by contact, the blocking element 315 into the hole 323 in the indexing spindle 307, as is shown in FIG. 12. When the control rod 327 is in the pushed-in position (see FIG. 12), the “quick coupling” balls 329 can move toward the inside of the tube 325 and disengage from the toroidal groove 331 cut into the arm 5. Thus, the assembly comprising the tube 325 and the control rod 327 (and thereby the support device/carriage supporting the source 9) can be moved toward the end of the arm.

As FIG. 13 shows, when the assembly comprising the tube 325 and the control rod 327 is pulled toward the end (the outside) of the arm 5, the blocking member (the indexing finger) 305 and the blocking element (indexing pin) 315 are pushed by their return spring 317, 319 toward the outside of the fixed pivot 301 (or of the column) so that the indexing finger becomes lodged in the guide body (indexing socket) 306. The respective lengths of the blocking member (indexing finger) 305 and of the blocking element (indexing pin) 315 are such that the blocking member (indexing finger) 305 become housed in the guide body (indexing socket) 306 before the blocking element (indexing pin) 315 leaves its housing in the hole 323 of the indexing spindle 307.

Because of the shape of the “quick coupling” system which connects the rod 327 and the control tube 325, it is not possible to envision a relative movement between these two elements unless a groove 331 is present in the arm 5 to allow axial movement of the balls 329. The arm 5 includes a plurality of grooves 331.

The outward movement of the blocking member (indexing finger) 305 is rendered possible in this horizontal position through the fact that the retaining element 309 or the retaining roller is situated, when the arm is in a horizontal position or substantially perpendicular to the column, facing a groove 331 in the retaining cam 311 which allows the retaining element (roller) 309 and, therefore, the blocking member (finger) 305 to move (the retaining element (roller) 309 and the blocking member (indexing finger) 305 are connected by a spindle).

The reverse movement of the support device (carriage)/source 9 assembly and the blocking of the rotation of the arm 5 are performed in the reverse order to the one described previously.

It will be appreciated that various modifications and/or improvements obvious to a person skilled in the art may be made to the various embodiments of the invention which have been described in the present description, without departing from the scope of the invention as defined by the attached claims.

The radiography appliance according to the present invention may comprise the anti-rotation mechanism 11, or the braking mechanism 100a, 100b or the anti-rotation AR and anti-displacement AD mechanism. The radiography appliance according to the present invention may also comprise the anti-rotation mechanism 11, and the braking mechanism 100a, 100b and the anti-rotation AR and anti-displacement AD mechanism.

1-56. (canceled)

57. A radiography device comprising:
a support column;
an arm configured to move and articulate on the support column, the arm including a first support device configured to bear a radiation source and a second support device configured to bear a radiography detector, at least one of the first and the second support device configured to move along the arm; and
anti-rotation device configured to limit or prevent rotation of the arm with respect to the support column.

58. The radiography device as claimed in claim 57, wherein the anti-rotation device is configured to differentially limit the rotation of the arm according to a position of the arm along the support column.

59. The radiography device as claimed in claim 57, wherein the anti-rotation device is configured to limit or prevent the rotation of the arm on a limited part of the support column.

60. The radiography device as claimed in claim 57, wherein the anti-rotation device includes:
a guide element extending over a limited part of the support column to guide the movement of the arm between a lower position in which the rotation of the arm with respect to the support column is limited and the arm is kept in a position substantially perpendicular to the support column, and an upper position allowing the rotation of the arm toward a position substantially parallel to the support column.

61. The radiography device as claimed in claim 57, wherein the anti-rotation device includes a limit stop to halt the movement of the arm at a minimum height.

62. The radiography device as claimed in claim 60, wherein the guide element includes an angular section allowing the rotation of the arm to be increased.

63. A radiography device comprising:
a support column;
an arm configured to move and articulate on the support column, the arm including a first support device configured to bear a radiation source and a second support device configured to bear a radiography detector, at least one of the first and the second support device configured to move along the arm; and
a braking device configured to slow a movement of the arm along the support column including a one-way braking system allowing the movement of the arm along the support column to be at least one of slowed and blocked.

64. The radiography device as claimed in claim 63, wherein the one-way braking system includes:
a first block;
a second block; and
a friction body,
wherein the first and second blocks are situated on each side of a rail of the support column, each one of the first and the second block delimiting a braking passage and a preloading passage in which the friction body lies.

65. The radiography device as claimed in claim 64, wherein the one-way braking system includes:
a preloading device applying a preloading force to each friction body to push each friction body toward an outside of the preloading passage and toward the braking passage and allowing self-locking braking.

66. The radiography device as claimed in claim 65, wherein the preloading device is configured to apply a force to the friction bodies to ensure that the one-way braking system is free of play.

67. The radiography device as claimed in claim 66, wherein the preloading device is configured to apply a force in a direction that is opposite of a direction of the preloading force to the friction bodies when the arm is moved along the rail in a first direction.

68. The radiography device as claimed in claim 67, wherein the preloading device is configured to apply a force in the same direction of the preloading force to the friction bodies when the arm is moved along the rail in a second direction the opposite of the first direction.

69. The radiography device as claimed in claim 64, wherein a braking passage is smaller than the preloading passage.

70. The radiography device as claimed in claim 57, further comprising:
a system configured to block the movement of the first support device or second support device depending on the inclination of the arm.

71. The radiography device as claimed in claim 57, further comprising:
an anti-rotation device configured to prevent a rotation of the arm with respect to the support column;
an anti-displacement device configured to prevent the movement of the first support device or the second support device along the arm; and
an actuator configured to activate the anti-rotation device and deactivate the anti-displacement device to allow the first or second support device to move along the arm without rotation.

72. The radiography device as claimed in claim 71, wherein the actuator is further configured to deactivate the anti-rotation device and activate the anti-displacement device to allow a rotation of the arm without movement of the first or second support device along the arm.

73. The radiography device as claimed in claim 71, wherein the actuator is configured to move along the arm to act on the anti-rotation device to activate and deactivate the same.

74. The radiography device as claimed in claim 71, wherein the anti-rotation device includes:
a blocking member configured to move between a first blocking position in which at least part of the blocking member is received by the arm and at least part of the blocking member is simultaneously received by part of the support column; and
a second non-blocking position in which the blocking member is housed entirely in part of the support column.

75. The radiography device as claimed in claim 74, wherein the arm is kept in a position substantially perpendicular to the support column when the blocking member is in its first blocking position.

76. The radiography device as claimed in claim 75, wherein the blocking member includes:
a blocking element configured to move between a first blocking position in which at least part of the blocking element is held in the blocking member and at least part of the blocking element is simultaneously received by the arm and a second non-blocking position in which the blocking element is housed entirely in the blocking member.
